(19) United States
(12) Patent Application Publication
(10) Pub. No.: US 2024/0269398 A1
(43) Pub. Date: Aug. 15, 2024

(54) SPRAY HEAD FOR A DEVICE FOR DISPENSING A FLUID PRODUCT

(71) Applicant: APTAR FRANCE SAS, Le Neubourg (FR)

(72) Inventors: Francois LE MANER, La Vallee Montaure (FR); Ludovic PETIT, Vitot (FR)

(73) Assignee: APTAR FRANCE SAS, Le Neubourg (FR)

(21) Appl. No.: 18/570,507

(22) PCT Filed: Jun. 15, 2022
(86) PCT No.: PCT/FR2022/051152

§ 371 Date: Dec. 14, 2023

(30) Foreign Application Priority Data
Jun. 17, 2021 (FR) FR2106463

(51) Int. Cl. A61M 11/00; A61M 15/08; A61M 39/24

(52) U.S. Cl. CPC A61M 11/007; A61M 15/08; A61M 39/24; A61M 2039/242

(57) ABSTRACT

A spray head (10) for a device for dispensing a fluid product, comprising a body (11) provided with a spray hole (12), the spray head (10) comprising a connecting member (15) rigidly attached to the body (11), the connecting member (15) defining a central axial channel (16), the spray head (10) comprising at least one valve (20; 40) forming precompression means for generating a calibrated spray through the spray hole (12), an intermediate member (14) being inserted between the connecting member (15) and the body (11), the intermediate member (14) being hollow and comprising an opening (14′) that opens into the connecting member (15), a sleeve (40) being mounted so as to slide in the connecting member (15) around the intermediate member (14) between an open position and a closed position, a resilient element (41) biasing the sleeve (14) to its closed position in which it closes off the opening (14′), the sleeve (40) being moved towards its open position when the flow of fluid product (F) is actuated based on a predetermined pressure.

The present invention relates to a spray head, in particular of the nasal type, and a device for dispensing fluid product comprising such a spray head.

Spray heads for devices for dispensing fluid product are well-known. Generally, spray heads are assembled on piston rods or valve members so as to guide the product, which is dispensed outside of a pump or a valve, to the spray hole. However, it has also been proposed to arrange a spray head directly on the outlet of a syringe-type reservoir so as to spray the content of said syringe rather than inject it through a needle. Document WO0071263 describes such a device.

Other devices of the prior art are described in documents WO0143794, EP3243571, WO2021025664, EP1092447 and WO03024519.

A disadvantage of a spray head assembled on a syringe-type reservoir relates to risks of re-suctioning liquid towards the inside of the reservoir, in particular due to a depression generated in the reservoir.

The present invention aims to provide a spray head which prevents any re-suctioning of liquid towards the reservoir, in particular at the end of actuation.

Another disadvantage existing with certain spray heads relates to the quality of the spray generated, which can depend on the actuation performed by the user, in particular the speed and the actuation force. To guarantee a good reproducibility of the spray, it is desirable to generate a calibrated spray, i.e. the properties of which are substantially similar independently of the way in which the user actuates the device.

The present invention also aims to provide a spray head which makes it possible to perform a correct and reproducible spraying.

Generally, the present invention aims to provide a spray head for a device for dispensing fluid product, which does not reproduce the abovementioned disadvantages.

The present invention also aims to provide such a spray head which is simple and inexpensive to manufacture and to assemble.

The present invention therefore aims for a spray head for a device for dispensing fluid product, comprising a body provided with a spray hole, said spray head comprising a connecting member rigidly attached to said body, said connecting member defining a central axial channel, said spray head comprising at least one valve forming precompression means making it possible to generate a calibrated spray through said spray hole, an intermediate member being interposed between said connecting member and said body, said intermediate member being hollow and comprising an opening that opens into said connecting member, a sleeve being mounted so as to slide into said connecting member around said intermediate member between an open position and a closed position, a resilient element, such as a spring, biasing said sleeve towards its closed position wherein it closes off said opening, said sleeve being moved to its open position when the flow of fluid product is actuated based on a predetermined pressure.

Advantageously, at least one valve forms anti-re-suctioning means preventing any re-suctioning of liquid towards said reservoir.

According to a first advantageous variant, the same valve forms said re-suctioning means preventing any re-suctioning of liquid towards said reservoir and said precompression means making it possible to generate a calibrated spray through said spray hole.

According to a second advantageous variant, a first valve forms said anti-re-suctioning means preventing any re-suctioning of liquid towards said reservoir and a second valve forms said precompression means making it possible to generate a calibrated spray through said spray hole.

Advantageously, said central axial channel of said connecting member comprises a valve comprising a valve seat engaging with a valve element which is movable and/or deformable between a rest position and an actuating position.

Advantageously, said valve element engages in the actuating position with a valve seat comprising axial projections separated by at least one groove, the fluid product being able to pass around said valve element and in said at least one groove to flow towards said spray hole.

Advantageously, said valve element engages in the actuating position with a valve seat comprising axial projections separated by at least one groove, the fluid product being able to pass around said valve element and in said at least one groove to flow towards said spray hole.

Advantageously, said valve element and said valve seat are produced such that the movement and/or the deformation of said valve element to its actuating position occurs only from a predetermined pressure of the flow of fluid product.

Advantageously, said valve element engages in its rest position sealingly with an internal shoulder of said central axial channel and is movable and/or deformable towards its actuation position.

The present invention also aims for a device for dispensing fluid product, comprising a syringe and a spray head such as described above, said spray head being assembled on said syringe.

Advantageously, said spray head comprises fixing means which engage with a standard Luer-type fixing provided on a front end of said syringe.

Advantageously, said fixing means comprise at least one external radial projection adapted to engage with an internal screw thread of said standard Luer-type fixing.

Advantageously, said syringe contains fluid product, a piston rigidly attached to an actuating rod being provided to dispense said fluid product.

These advantages and features and others of the present invention will appear more clearly during the following detailed description, made in reference to the accompanying drawings, given as non-limiting examples, and wherein

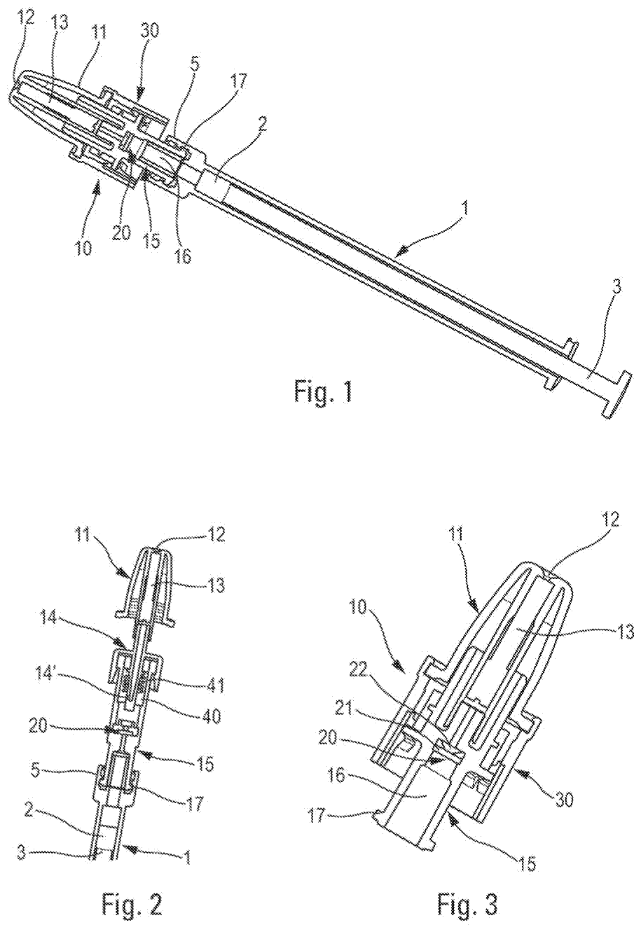

FIG. 1 is a schematic, cross-sectional view of a spray head according to a first embodiment, assembled on a syringe-type reservoir,

FIG. 2 is a view similar to that of FIG. 1, showing a spray head according to an embodiment of the invention,

FIG. 3 is a schematic, cross-sectional view of the spray head of FIG. 1,

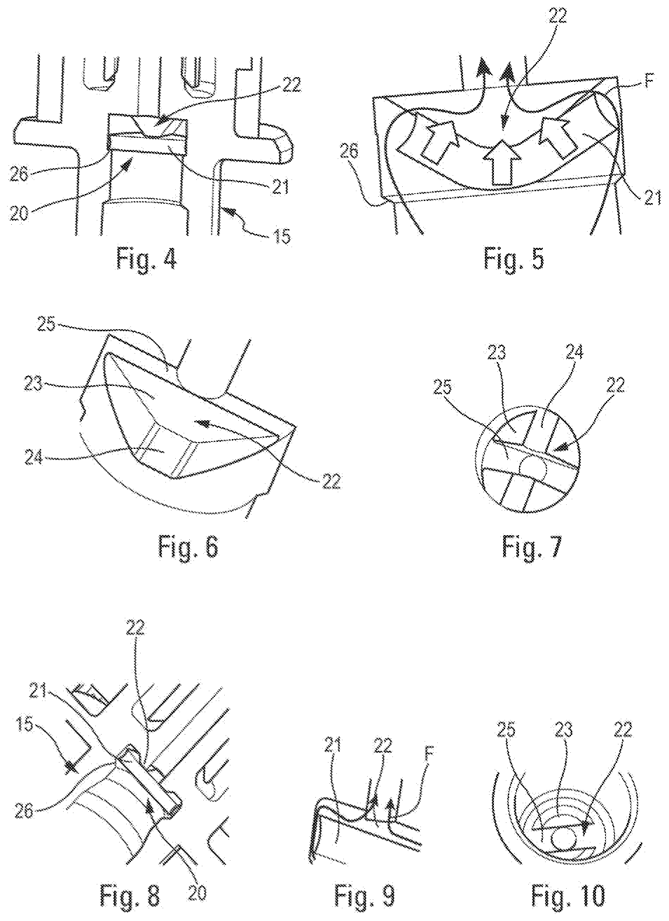

FIG. 4 is a schematic, detailed view of a valve part of the spray head of FIGS. 1 and 3, before and/or after actuation,

FIG. 5 is a view similar to that of FIG. 4, during actuation,

FIG. 6 is a detailed, perspective, cross-sectional view of the valve seat of FIGS. 4 and 5,

FIG. 7 is a detailed bottom view of the valve seat of FIGS. 4 and 5,

FIG. 8 is a view similar to that of FIG. 4, illustrating a valve part according to a second embodiment,

FIG. 9 is a detailed view of FIG. 8, during actuation, and

FIG. 10 is a detailed bottom view of the valve seat of FIGS. 8 and 9.

The present invention will be described below in reference to three embodiments, but it is understood that other embodiments could be considered.

FIG. 1 represents a spray head 10 assembled on a syringe-type reservoir 1. Preferably, the reservoir 1 contains a single dose of fluid product, generally a liquid, which is dispensed in one single actuation. The present invention therefore mainly relates to single-use devices, and the description below will be made in reference to a reservoir formed by a single-dose syringe. It must however be noted that the reservoir could contain more than one dose, for example two doses, with dose-splitting means to dispense the content of the reservoir in several successive actuations.

Conventionally, suitable dispensing means can be provided in said syringe 1 to dispense its content. Typically, these dispensing means comprise a piston 2 rigidly attached to an actuating rod 3 actuated by the user at the time of dispensing. Naturally, different dispensing means could be considered, for example automatic dispensing means, of the auto-injector type. However, these dispensing means not intervening directly in the present invention, they will not be described in more detail below.

The spray head 10 comprises a body 11 provided with a spray hole 12. Preferably, as represented in the drawings, the spray head 10 is of the nasal type, with an extended body 11 to penetrate into a nostril and the spray hole 12 disposed at the distal axial end of said body, in the flow direction of the fluid product during actuation. Advantageously, an insert 13 is disposed upstream of the spray hole 12 to generate a spray, for example by means of a well-known spray profile (not represented in the drawings).

The body 11 is rigidly attached to a connecting member 15, which defines a central axial channel 16 and which comprises fixing means 17 to be fixed onto a reservoir 1, in particular a syringe body as represented in FIG. 1. Preferably, the body 11 is fixed to the connecting member 15, but these two parts could also be formed of one single one-piece part, for example by additive printing.

Advantageously, the fixing means 17 are adapted to engage with a standard Luer-type fixing 5 provided on the front end of the syringe 1. In a well-known manner, and as can be seen in FIG. 1, these fixing means 17 comprise at least one external radial projection adapted to engage with an internal screw thread (not represented) of said standard Luer-type fixing 5 of the syringe 1.

To avoid the spray head 10 being able to be easily dismounted from the syringe 1, the spray head 10 can advantageously comprise an external ring g 30 mounted rotatably around the connecting member 15 in the same way as a ratchet. During the assembly of the spray head 10 on the syringe 1, the external ring 30 is rotated clockwise, which makes it possible to screw the connecting member 15 in the standard Luer-type fixing 5 of the syringe 1. When the external ring 30 is rotated anticlockwise, it rotates into the vacuum, thus preventing dismounting the spray head 10 from the syringe 1.

According to the invention, the spray head comprises at least one valve 20, 40 forming precompression means making it possible to generate a calibrated spray through the spray hole 12.

In the first embodiment, represented in FIGS. 1 and 3 to 7, the central axial channel 16 comprises a valve 20 comprising a valve seat 22 engaging with a valve element 21 which is movable and/or deformable between a rest position and an actuating position.

The valve element 21 is made of deformable material, for example rubber, and engages in the actuating position with a valve seat 22 comprising truncated-shaped axial projections 23, each provided with a flat peak 24 and separated by at least one groove 25. When the device is actuated, the fluid product which flows along the arrow F in FIG. 5 deforms the valve element 21 from its rest position, which can be seen in FIG. 4, to its actuating position, which can be seen in FIG. 5. In the actuating position, the valve element is folded and is flattened on the valve seat 22, with the fluid being able to pass around the valve element 21 and in the at least one groove 25 to flow towards the spray hole 12. The valve element 21 and the valve seat 22 are produced such that the deformation of the valve element 21 occurs only from a pressure of the predetermined flow F of liquid. As long as this predetermined pressure is not reached, the valve element 21 is not deformed towards its actuating position. Thus, whatever the speed or the actuating force exerted by the user, the precompression generated by the valve 20 makes it possible to guarantee a calibrated and reproducible spray, independently from the way in which the user actuates the device. By calibrated spray, this means a spray that has substantially the same properties upon each actuation, in particular the ejection speed of the spray, the form of the spray and the size of the droplets.

FIGS. 8 to 10 illustrate another embodiment, wherein the valve element 21 can be axially moved under the effect of the flow F of liquid. At rest, it rests on an internal shoulder 26 of the central axial channel 16, and sealingly closes the passage towards the reservoir 1. When the flow F of liquid arrives, the valve element 21 is moved towards the valve seat 22 to enable the expulsion of the liquid. In this case, the valve seat 22 also comprises axial projections 23 separated by at least one groove 25, and during actuation, the valve element 21 is flattened on the valve seat 22, with the liquid being able to pass around the valve element 21, and in the at least one groove 25 to flow towards the spray hole 12. At the end of actuation, the valve element 21 returns into the rest position, in particular, by gravity. In this case, in case of depression in the reservoir 1 at the end of actuation, the valve element 21 will be flattened on the internal shoulder 26 and thus prevent any pre-suctioning of liquid towards the reservoir 1.

It must be noted that the embodiment of FIGS. 4 to 7 can also incorporate this anti-re-suctioning function, with an internal shoulder 26 engaging with the valve element 21 at rest and in case of depression in the reservoir 1, as can be seen in FIGS. 4 and 5. In this case, one single valve 20 is sufficient in the spray head to perform both the precompression function and the anti-re-suctioning function.

FIG. 2 illustrates an embodiment according to the invention, wherein there are two valves, a first valve 20 to perform the anti-re-suctioning function and a second valve 40 to perform the precompression function.

The first valve 20 can be identical to that described above in reference to FIGS. 8 to 10.

The second valve 40 is formed by a sleeve 40 mounted so as to slide between an open position and a closed position in said connecting member 15 around an intermediate member 14. This intermediate member 14 is interposed between the connecting member 15 and the body 11. The intermediate member 14 is hollow and comprises an opening 14′ that opens, in particular laterally, into said connecting member 15. A resilient element 41, such as a spring, biases said sleeve 14 to its closed position, wherein it closes off said opening 14′, said sleeve 40 being moved to its open position when the flow of fluid product F is actuated based on a predetermined pressure. In this case, it is therefore the spring 41 which determines the precompression threshold above which the valve opens. Optionally, the second valve 40 could also act as an anti-re-suctioning valve, and in this case, the single valve 40 would be sufficient to perform both the precompression function and the anti-re-suctioning function.

Although the present invention has been described above with reference to several embodiments thereof, it is understood that the invention is not limited to the embodiments described and represented in the drawings, but, on the contrary, any useful modification could be applied thereto by a person skilled in the art, without going beyond the scope of the present invention, such as defined by the accompanying claims.

1. Spray head for a device for dispensing fluid product, comprising a body provided with a spray hole, said spray head comprising a connecting member rigidly attached to said body, said connecting member defining a central axial channel, said spray head comprising at least one valve forming precompression means making it possible to generate a calibrated spray through said spray hole, characterised in that an intermediate member is interposed between said connecting member and said body, said intermediate member being hollow and comprising an opening that opens into said connecting member, a sleeve being mounted so as to slide into said connecting member around said intermediate member between an open position and a closed position, a resilient element, such as a spring, biasing said sleeve towards its closed position, wherein it closes off said opening, said sleeve being moved to its open position when the flow of fluid product is actuated based on a predetermined pressure.

2. Head according to claim 1, wherein at least one valve forms anti-re-suctioning means preventing any re-suctioning of liquid towards said reservoir.

3. Head according to claim 2, wherein a first valve forms said anti-re-suctioning means preventing any re-suctioning of liquid towards said reservoir and a second valve forms said precompression means making it possible to generate a calibrated spray through said spray hole.

4. Head according to claim 1, wherein said central axial channel of said connecting member comprises a valve comprising a valve seat engaging with a valve element which is movable and/or deformable between a rest position and an actuating position.

5. Head according to claim 4, wherein said valve element engages in the actuating position with a valve seat comprising axial projections separated by at least one groove, the fluid product being able to pass around said valve element and in said at least one groove to flow towards said spray hole.

6. Head according to claim 5, wherein said valve element engages in the actuating position with a valve seat comprising axial projections separated by at least one groove, the fluid product being able to pass around said valve element and in said at least one groove to flow towards said spray hole.

7. Head according to claim 6, wherein said valve element and said valve seat are produced such that the movement and/or the deformation of said valve element towards its actuating position occurs only from a predetermined pressure of the flow of fluid product.

8. Head according to claim 4, wherein said valve element engages in its rest position sealingly with an internal shoulder of said central axial channel and is movable and/or deformable towards its actuating position.

9. Device for dispensing fluid product, comprising a syringe and a spray head according to claim 1, said spray head being assembled on said syringe.

10. Device according to claim 9, wherein said spray head comprises fixing means which engage with a standard Luer-type fixing provided on a front end of said syringe.

11. Device according to claim 10, wherein said fixing means comprise at least one external radial projection adapted to engage with an internal screw thread of said standard Luer-type fixing.

12. Device according to claim 9, wherein said syringe contains fluid product, a piston rigidly attached to an actuating rod being provided to dispense said fluid product.
